(19) United States
(12) Patent Application Publication
(10) Pub. No.: US 2024/0266049 A1
(43) Pub. Date: Aug. 8, 2024

(54) PRIVACY-PRESERVING INTERPRETABLE SKILL LEARNING FOR HEALTHCARE DECISION MAKING

(71) Applicant: NEC Laboratories America, Inc., Princeton, NJ (US)

(72) Inventors: Wenchao Yu, Plainsboro, NJ (US); Haifeng Chen, West Windsor, NJ (US); Wei Cheng, Princeton Junction, NJ (US)

(21) Appl. No.: 18/425,715

(22) Filed: Jan. 29, 2024

Related U.S. Application Data
(60) Provisional application No. 63/442,475, filed on Feb. 1, 2023.
(60) Provisional application No. 63/526,702, filed on Jul. 14, 2023.

(51) Int. Cl. G16H 50/20; G16H 40/20

(52) U.S. Cl. CPC G16H 50/20; G16H 40/20

(57) ABSTRACT

Methods and systems for training a healthcare treatment machine learning model include aggregating local weights from a set of clients to update a set of global weights for an imitation-based skill learning model. A set of local prototype vectors are clustered from the plurality of clients to generate clusters. Representative vectors are selected for the clusters as a set of global prototypes. Client-specific prototype vectors are determined for the clients based on the representative vectors. The updated set of global weights and the client-specific prototype vectors are distributed to the clients.

### RELATED APPLICATION INFORMATION

This application claims priority to U.S. Patent Application No. 63/442,475, filed on Feb. 1, 2023, and to U.S. Patent Application No. 63/526,702, filed on Jul. 14, 2023, each incorporated herein by reference in its entirety.

### BACKGROUND

### Technical Field

The present invention relates to machine learning systems and, more particularly, to interpretable skill learning via imitation.

### Description of the Related Art

Imitation learning is a type of machine learning technique that replicates experts' skills via their demonstrations. Imitation is useful in decision-making tasks, but certain challenges limit its applicability in real-world scenarios. For example, imitation learning systems may lack intrinsic interpretability to explicitly explain the underlying rationale of the learned skill, making the learned policy difficult to trust by human operators. Furthermore, due to the scarcity of expert demonstrations for specific contexts, learning a policy based on different data silos may be needed. However, sharing such data in privacy-sensitive applications, such as finance and healthcare, may be difficult or impossible due to regulatory and practical limitations.

### SUMMARY

A method for training a healthcare treatment machine learning model includes aggregating local weights from a set of clients to update a set of global weights for an imitation-based skill learning model. A set of local prototype vectors are clustered from the plurality of clients to generate clusters. Representative vectors are selected for the clusters as a set of global prototypes. Client-specific prototype vectors are determined for the clients based on the representative vectors. The updated set of global weights and the client-specific prototype vectors are distributed to the clients.

A system for training a healthcare treatment machine learning model includes a hardware processor and a memory that stores a computer program. When executed by the hardware processor, the computer program causes the hardware processor to aggregate local weights from a plurality of clients to update a set of global weights for an imitation-based skill learning model, to cluster a set of local prototype vectors from the plurality of clients to generate a plurality of clusters, to select representative vectors for the plurality of clusters as a set of global prototypes, to determine client-specific prototype vectors for the plurality of clients based on the representative vectors, and to distribute the updated set of global weights and the client-specific prototype vectors to the plurality of clients.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of a system for federated training of a skill learning model, in accordance with an embodiment of the present invention;

FIG. 2 is a block diagram of a client that performs local imitation learning, in accordance with an embodiment of the present invention;

FIG. 3 is pseudo-code of a method for training an interpretable skill learning model, in accordance with an embodiment of the present invention;

FIG. 4 is pseudo-code of a method for local prototype projection, in accordance with an embodiment of the present invention;

FIG. 5 is a block diagram of a healthcare facility that uses a skill imitation system to guide patient treatment, in accordance with an embodiment of the present invention;

FIG. 6 is a diagram of patient treatment guided by actions selected by a skill imitation system, in accordance with an embodiment of the present invention;

FIG. 7 is a block diagram of a method of federated training of a skill learning model, in accordance with an embodiment of the present invention;

FIG. 8 is a block diagram of computing device that can perform federated training of a skill learning model, in accordance with an embodiment of the present invention;

FIG. 9 is an exemplary neural network architecture that can be used as part of an imitation learning layer, in accordance with an embodiment of the present invention; and

FIG. 10 is an exemplary deep neural network architecture that can be used as part of an imitation learning layer, in accordance with an embodiment of the present invention.

### DETAILED DESCRIPTION OF PREFERRED EMBODIMENTS

Interpretable skill learning may be implemented using a global policy that incorporates data from different sources and that provides explainable interpretations to each local user, without violating privacy and data sovereignty. Interpretable skill learning can capture the varying patterns in the trajectories of expert demonstrations and can extract prototypical information as skills that provide implicit guidance for policy learning and explicit explanations in the reasoning process. An aggregation mechanism is coupled with the skill-based learning model to preserve global information utilization and to maintain local interpretability under a federated framework.

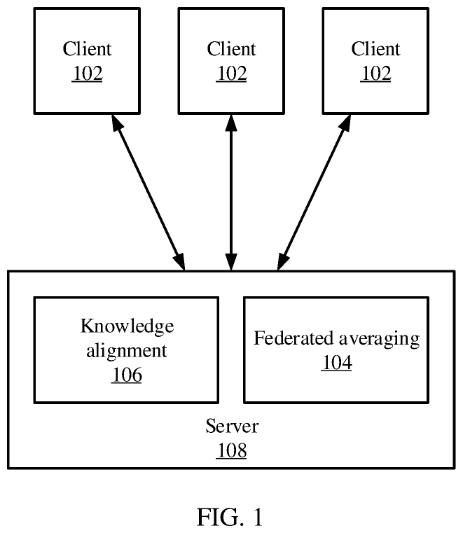

Referring now to FIG. 1, a diagram of a federated interpretable learning framework is shown. A set of clients C includes |C| individual clients 102. Each client aims to learn an interpretable skill learning model that includes a client-specific prototype layer that connects the other learning components that generate a segment representation and that perform an imitation learning task. A set of parameterized vectors in the prototype layer learn from segment representations to establish the prototypes and to construct skill embeddings, providing contextual information for imitation learning.

At a server 108, the client models are aggregated by federated averaging 104 and knowledge alignment 106 to align similar prototypical information. After global and local training, parameterized vectors of each client 102 may be translated to interpretable prototypes via the association in the representation space of local training data. Each client can explain the skills it employs based on the most similar prototypes it uses in its reasoning process.

The server 108 initializes and distributes the global interpretable imitation-based skill learning model to local clients 102 at the beginning of global training. The model may be partitioned into two sets of parameters—one set that includes the parameterized vectors of the prototype layer, and another that includes the weights of the convolutional layer 202 and the imitation learning layer 204.

Federated training is performed at each global epoch, aggregating the local models from clients 102 after local training. Federated averaging 104 may be used to aggregate the local models to generate a segment representation and final action. Knowledge alignment 106 is performed to correct parameterized vector sets that differ between clients 102 due to the heterogeneity of expert demonstrations across the different clients.

Knowledge alignment 106 may include clustering to identify group membership of each parameterized vector, for example using K-means and Gaussian mixture models with K groups. The vectors with similar prototypical/skillful representations are aligned to the same clusters. Each local vector may be matched by the global centroid vector of a group, based on the identified membership and may be distributed back to local clients. As a result, each client 102 owns a specific vector set that best represents the prototypical information from its own data through the entire process. Skillful knowledge is shared and aligned across different clients 102.

Although the present embodiments are described with specific focus on applications in the field of medical decision-making, it should be understood that the present embodiments may be applied to a wide variety of different circumstances. For example, the clients 102 may represent autonomous vehicles, where the action policy dictates maneuvering actions for the vehicle to take based on sensed information from the vehicle's environment.

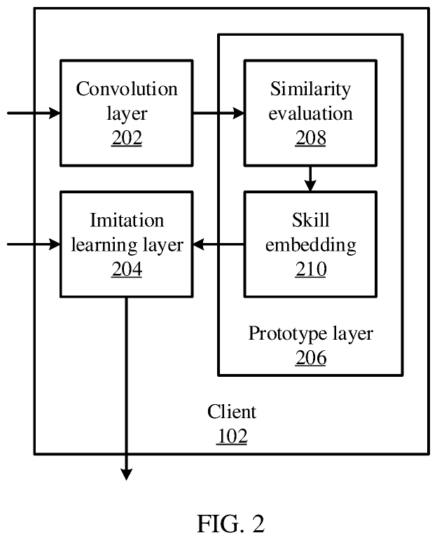

Referring now to FIG. 2, additional detail is shown on a client 102. Input regarding a sequence is provided to a convolutional layer 202 and to an imitation learning layer 204. The convolutional layer 202 processes the segments and generates a representation vector rₜ for each time step t. This representation is compared to a set of prototypes at similarity evaluation 208, which generates a vector of similarity values. These similarity values are converted to a skill vector eₜ using the prototypes, and the embedded skill is passed to imitation learning layer 204.

The imitation learning layer 204 is provided a current time step sₜ in the sequence. The imitation learning layer 204 thus generates an action aₜ responsive to the current state sₜ that applies a skill based on the sequence of previous states. Because the action is based on the set of prototypes, it can be determined which prototypes most influenced that action. These prototypes may be based on predetermined skills and conditions, so that the prototypes which are selected can be used to provide a rationale for why the particular action was selected.

The input data may represent a sequence of states for each client 102. The input trajectory may be divided into non-overlapping segments of length m. Each state may come with the previous m−1 states from the same trajectory, encoding temporal dynamics up to the current time step t. Skill transitions across consecutive segments are captured by the learning model. Padding the initial state may be performed during exploitation when needed.

The convolutional layer 202 encodes the temporal dynamics from the input segment. It is contemplated that a convolution-based, recurrent-based, or transformer-based encoder may be used. A convolution-based encoder may be particularly useful for extracting salient information from segments with short lengths.

The contextual imitation learning layer 204 determines an action to be performed. A contextual policy is built based on behavior cloning, learning a mapping from state to action in a supervised manner.

The prototype layer 206 generates the skill embedding. Parameterized vectors are used to reconstruct the skill information preserved by the segment representation, which renders a flexible interpretation structure. During reasoning, all parameterized vectors are projected to prototypes that are the segment representations of local training data. The underlying skill of a segment is explained by similar prototypes having high weights.

The input segment at a time step t may be represented as [sₜ₋ₘ₊₁> . . . , sₜ₋₁, sₜ)∈ [symbol] ^{m×d}, where d is the feature dimension of each state s and m is a number of time steps. A representation rₜ may be extracted using the two-dimensional convolutional layer 202 which encodes the feature and the temporal dynamics:

$${r}_{t} = Conv ( {s}_{t - m + 1 : t} ) = tanh ( {CAT}_{i = 0}^{h - 1} ( {W}_{i} ★ {s}_{t - m + 1 : t} + {b}_{i} ) )$$

where Wᵢ is a weight term for the kernel of the convolutional layer 202, bᵢ is a bias term for the kernel of the convolutional layer 202, * is a two-dimensional cross-correlation operator, h is a number of kernels, and CAT(⋅) is a concatenation function.

The prototype layer 206 makes use of a set of k parameterized prototype vectors, P=[p₁, . . . pₖ]∈ [symbol] ^{k×h}, having dimensionality equal to rₜ. Each prototype vector is optimized to be close to a set of similar segments in the representation space. During a forward pass, the similarity between a segment representation rₜ and each parameterized prototype vector is first evaluated by an exponential function based on an L2 distance:

$${e}^{- {‖ {r}_{t} - p ‖}_{2}^{2}}$$

which bounds the similarity to a unit range. To measure the relative similarity, all evaluated scores are re-scaled by their sum, and the final pairwise similarity score for the segment representation and the i^{th} vector is:

$${sim}_{{r}_{t} , {p}_{i}} = \frac{{e}^{- {‖ {r}_{t} - {p}_{i} ‖}_{2}^{2}}}{{Σ}_{i = 1}^{k} {e}^{- {‖ {r}_{t} - {p}_{i} ‖}_{2}^{2}}}$$

All relative similarity scores are concatenated to a similarity vector Wₛᵢₘ∈ [symbol] ^{k×1} as Wₛᵢₘ=[sim₍ᵣ_{ₜ}_{,p}_{₁}₎, sim₍ᵣ_{ₜ}_{,p}_{₂}₎ . . . , sim₍ᵣ_{ₜ}_{,p}_{_{K}}₎], which imply the importance of the corresponding vectors in the skill embedding generation.

Thus, the skill embedding eₜ∈ [symbol] ^{1×h} may be generated by the weighted combination of all parameterized vectors:

$${e}_{t} = {W}_{sim}^{T} \cdot P$$

The segment representation rₜ is not directly used in the final form of a skill embedding, as it may not be interpretable. Instead, the parameterized prototype vectors may be used to reconstruct the skill information preserved by the segment representation. In the reasoning process, all prototype vectors p are projected to prototypes that are the segment representations of local training data, and the skill may be explained by reference to similar prototypes. This follows a sort of soft-skill combination, which can be altered to a hard-skill selection by adopting a Gumbel-Softmax mechanism.

The imitation learning layer 204 builds a contextual policy based on behavior cloning to learn a mapping from an input state sₜ to an action aₜ in a supervised manner. The contextual policy is parameterized by θ and is denoted as π_{θ}(aₜ|eₜ, sₜ), which takes the concatenation of skill embedding and the state as input. The skill embedding captures varying patterns of expert trajectories and guides the agent to perform primitive actions more accurately:

$${a}_{t} ← {π}_{θ} ( {a}_{t} \vert {e}_{t} , {s}_{t} )$$

The model provides explicit interpretations to the varying patterns in the reasoning process.

Learning objectives may include a segment-level imitation learning objective and three objectives that reinforce the interpretability of the prototypes and skills on these non-overlapped segments. Each component is introduced below based on a batch of segments in a training dataset: {[(sₜ⁽ⁱ⁾,aₜ⁽ⁱ⁾)]ᵢ₌₁^{m}}ᵢ₌₁ⁿ, with a batch size n and a segment length m.

The first objective may be to minimize an imitation learning loss with behavior cloning:

$${ℒ}_{im} = {\sum }_{i = 1}^{n} {\sum }_{t = 1}^{m} {π}_{E} ( {a}_{t}^{( i )} \vert {s}_{t}^{( i )} ) log {π}_{θ} ( {a}_{t}^{( i )} \vert {e}_{t}^{( i )} , {s}_{t}^{( i )} )$$

where π_{E} denotes an expert policy that generates demonstrations.

The second objective regularizes the non-overlapped segment representation output from the convolution layer 202 to be as adjacent to its closest prototype as possible, which enforces a clustering structure of segments in the representation space. This is achieved by minimizing the smallest L2 distance between rₜ₌ₘ and all vectors in the set P:

$${ℒ}_{c} = {\sum }_{i = 1}^{n} {\sum }_{t = 1}^{m} {min}_{{p}_{j} ∈ P} {‖ {r}_{t = m}^{( i )} - {p}_{j} ‖}_{2}^{2}$$

A third objective reverse-regularizes each vector to be as similar to a segment representation as possible by minimizing the smallest L2 distance between each pᵢ and a batch of non-overlapping segment representations rₜ₌ₘ∈ [symbol] ₙ=[[Conv (s_{t-m+1:t}^{(j)}]ₜ₌ₘ]ⱼ₌₁ⁿ, which helps the downstream projection with evidencing segments:

$${ℒ}_{e} = {\sum }_{i = 1}^{k} {min}_{{r}_{t = m} ∈ {ℛ}_{n}} {‖ {p}_{i} - {r}_{t = m}^{( j )} ‖}_{2}^{2}$$

where [symbol] ₙ represents a collection of all non-overlapping segment representations in the training set, with each segment having a length m.

Two objectives impose dual regularization on the learning of the convolution layer 202 and the prototype layer 206 toward a clearer representation structure for interpretation.

A fourth objective enforces a diverse structure of learnable parameterized prototype vectors to avoid redundancy and to improve the generalizability of resulting prototypes, where the L2 distance between each pair of vectors is penalized, with a threshold dₘᵢₙ:

$${ℒ}_{d} = {\sum }_{i = 1}^{k} {\sum }_{j \neq i}^{k} max ( 0 , {d}_{m i n} - {‖ {p}_{i} - {p}_{{i}^{′}} ‖}_{2}^{2}$$

As such, the full objective function to be minimized can be written as [symbol] = [symbol] ᵢₘ+λ₁ [symbol] _{c}+λ₂ [symbol] ₑ+λ₃ [symbol] _{d}, with different weights λ₁, λ₂, and λ₃ balancing the components.

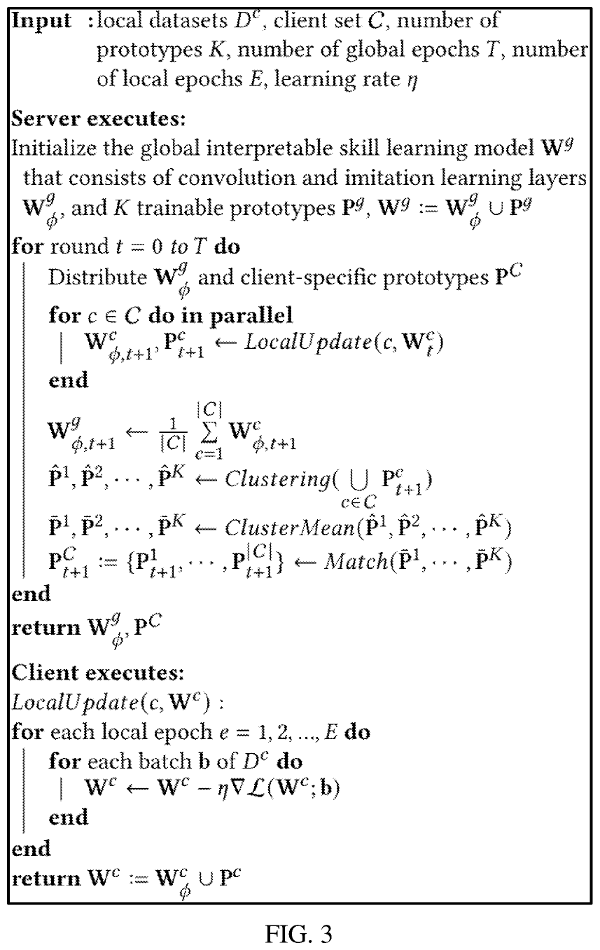

Referring now to FIG. 3, pseudo-code for federated training of a skill prediction model is shown. Global prototype alignment is performed via clustering during training, and a local prototype projection is performed at model deployment to enhance interpretability in a privacy-preserving manner.

A global server 108 initiates and distributes the global interpretable skill learning model to local clients 102 at the beginning of global training. The model is partitioned into two sets of parameters, one denoted as P^{g} that includes learnable vectors in the prototype layer 206, and the other denoted as W^{g} that includes the weights of the convolution layer 202 and the imitation learning layer 204.

For each global epoch, the server 108 aggregates the local models from clients 102 after local training. The weights W^{g} may be aggregated to generate segment representations and final actions. However, the learnable vector sets can differ due to the heterogeneity of expert demonstrations across different clients. The discrepancy leads to a misalignment between local and global skills if no corrective action is taken.

To this end, a prototype aggregation may be performed at the server 108 side. After receiving K×|C| vectors, where K is a number of prototypes and C is a client set, the server 108 performs clustering to identify the membership of each vector. Clustering may be performed by any appropriate clustering method, such as K-means or Gaussian mixture models. Vectors that have similar skill representations may be aligned to a same clusters.

After clustering, the centroid vector that represents a mode of skill is obtained by the mean of all vectors that belong to the same cluster. Each vector is matched by the centroid vector based on the identified membership and the vector sets are distributed to local clients. As such, each client 102 owns a specific vector set that best represents the skills from its own data through the entire training process and skill knowledge is shared across different clients. A matching function replaces each element in the local vectors with the centroid of their respective clusters, as determined by the identified cluster membership.

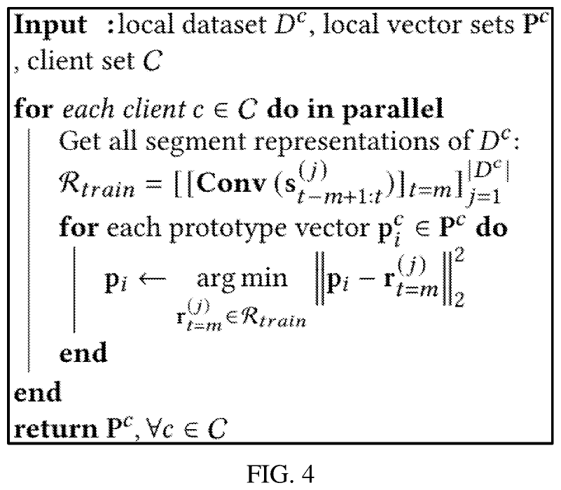

Referring now to FIG. 4, pseudo-code for local prototype projection is shown. After training is completed and the local vector set with skill representations is well-regularized, the returned skill learning model may not be readily interpretable. This is because the vectors are approximations and are not associated with real data that has explicit prototypes. To enable the interpretation of each local skill learning model, local prototype projection may be performed to each vector by assigning it to the training segment that has the smallest L2 distance in the representation space.

After this stage, the vectors are updated to explicit prototypes and the local model is able to capture the varying patterns to construct a meaningful skill embedding based on the prototypes. This step is privacy-sensitive, and so may be performed once on the local data during the inference and reasoning stage for each client 102, so that the privacy of expert demonstrations may be preserved and so that no data or data representations are leaked to the global server 108.

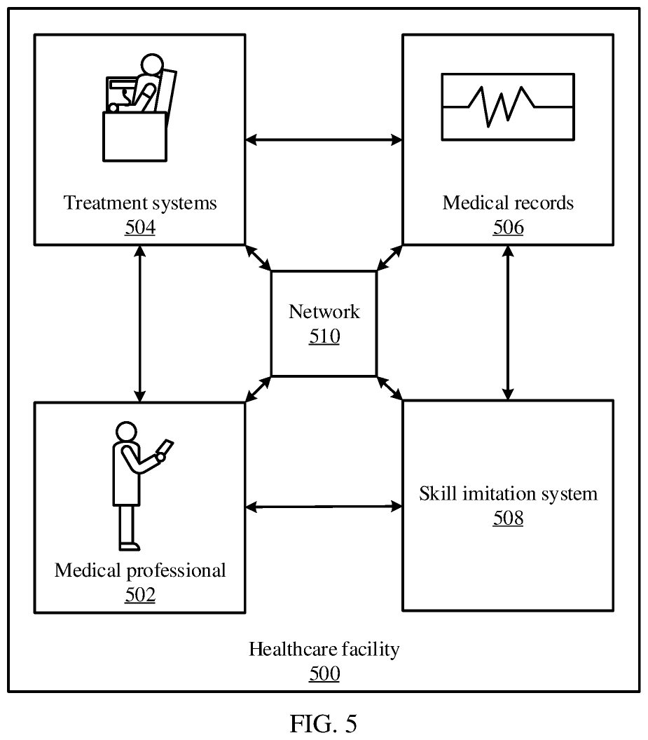

Referring now to FIG. 5, a diagram of skill learning is shown in the context of a healthcare facility 500. Imitation learning may be used to learn a mapping between states and actions when devising a treatment for a patient in a healthcare context. The imitation learning aims to replicate expert behavior, such as diagnosis and treatment actions performed by doctors, based on demonstrations from a set of records. To that end, an interpretable sequence modeling framework may be used to identify an expert's trajectory based on sequence data with temporal characteristics. Learning and inference may be performed at a segment level, which captures temporal variability of states and identifies skills that are transferable across different trajectories. An interpretable skill learning model is therefore provided to learn treatment policies, exploiting the segment-level expert demonstrations and results in representative, transferable skills across different trajectories. The prototypes described above represent distinct skills, in the form of treatment actions that can be performed in a medical context.

Imitation learning may be used to help monitor and treat multiple patients, for example responding to changes in their particular healthcare needs. The healthcare facility may include one or more medical professionals 502 who provide information relating to events and measurements of system status to skill imitation system 508. Treatment systems 504 may furthermore monitor patient status to generate medical records 506 and may be designed to automatically administer and adjust treatments as needed.

Based on information drawn from at least the medical professionals 502, treatment systems 504, and medical records 506, skill imitation system 508 learns skills applied by the medical professionals 502 in response to developing patient conditions. For example, the medical records 506 may include historical patient healthcare conditions (e.g., biometric information and a description of symptoms) and actions taken by the medical professionals 502 responsive to those conditions.

The different elements of the healthcare facility 500 may communicate with one another via a network 510, for example using any appropriate wired or wireless communications protocol and medium. Thus the skill imitation system 508 may access remotely stored medical records 506, may communicate with the treatment systems 504, and may receive instructions and send reports to medical professionals 502. In particular, the skill imitation system 508 may automatically trigger treatment changes for a patient, responsive to new information gleaned from the medical records 506, by sending instructions to the treatment systems 504.

In some cases, the skill imitation system 508 may generate a specific treatment plan for the patient, including a prescription plan that includes drugs that will help treat the patient, a meal provision plan to address the patient's dietary needs, a rehabilitation plan to provide for physical therapy and other activities needed for the patient to recover, and a discharge destination plan that indicates whether the patient may return home, should remain, or should be transferred to another healthcare facility. The output of the skill imitation system 508 may therefore include one or a combination of the above automatic treatments and plan outputs. In some cases, the treatment plan may be used by a medical professional to aid in decision-making for patient management.

In some cases, the healthcare facility 500 may represent a single client 102, with its records being used in-house for local training and with privacy-sanitized information being passed to a remote server 108 for federated learning. In some cases, the healthcare facility 500 may include multiple clients 102, with private information being siloed within such individual systems and with the server 108 being operated by the healthcare facility 500 as well.

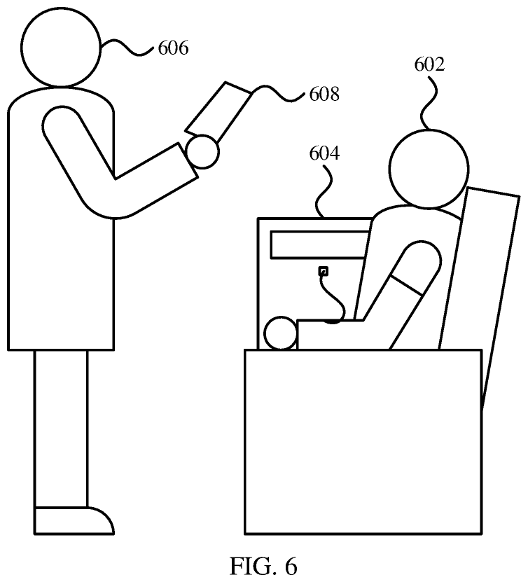

Referring now to FIG. 6, patient 602 is shown in the context of a healthcare system. For example, the patient 602 may be in a hemodialysis (also known simply as “dialysis”) session. During dialysis, a dialysis machine 604 automatically draws the patient's blood, processes and purifies the blood, and then reintroduces the purified blood to the patient's body. Dialysis can take as long as four hours to complete, and may be performed every three days, though other durations and periods are contemplated. Although dialysis is specifically contemplated, it should be understood that any appropriate medical procedure or monitoring may be used instead.

Before, during, and after a dialysis session, a patient 602 may experience a health event relating to the treatment. Such health events can be dangerous to the patient 602, but can be predicted based on knowledge of previous health events and the patient's present health metrics. The recommendation 608 may furthermore include information relating to the type of event that is predicted, as well as measurements of the patient's status It is specifically contemplated that this recommendation may be made before the dialysis session begins, so that treatment can be adjusted.

The recommendation may be made based on a variety of input information. Part of that information includes a static profile of the patient, for example including information such as age, sex, starting time of dialysis, previous health events, etc. The information also includes dynamic data, such as dialysis measurement records, which may be taken at every dialysis session, blood pressure, weight, venous pressure, blood test measurements, and cardiothoracic ratio (CTR). The blood test measurements may be taken regularly, for example at a frequency of twice per month, and may measure such factors as albumin, glucose, and platelet count. The CTR may also be taken regularly, for example at a frequency of once per month. Dynamic information may also be recorded during the dialysis session, for example using sensors in the dialysis machine 604. The dynamic information may be modeled as time series over their respective frequencies.

In addition, the systems themselves may be monitored within a healthcare environment. For example, operational parameters of a dialysis machine 604 or any other system in a hospital or other healthcare facility many be monitored, along with a history of past events at the system, to predict events as described below.

During treatment, the status of the patient 602 may be continuously monitored, for example tracking the patient's heart rate and other vital signs. In the event that the patient's vital signs indicate an imminent or ongoing adverse health event, the treatment may be altered accordingly. For example, the treatment systems may automatically administer a drug or shut down treatment responsive to a negative health event.

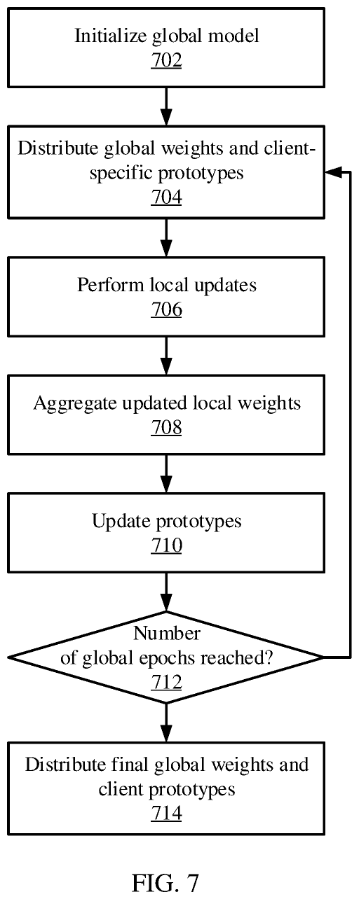

Referring now to FIG. 7, a method for training local skill prediction models is shown. Following the pseudo-code of FIG. 3, block 702 initializes the global model, including client set C, local datasets DE, number of prototypes K, number of global epochs T, number of local epochs E, and learning rate n. The global interpretable skill learning model W^{g} and the K trainable prototypes P^{g} are similarly initialized as W^{g}= [symbol] ∪P^{g}, with W_{ϕ}^{g} being the parameters of the convolution layer 202 and the imitation layer 204.

Block 704 distributes the global weights and client-specific prototypes from the server 108 to the clients 102. This information may be transmitted by any appropriate means. Block 706 then performs local updates of the parameters and prototypes at each of the clients 102, as shown in FIG. 3, with updated local parameters W^{C} being returned to the server 108.

At the server 108, the local weights are aggregated in block 708 to form an updated set of global weights. This may be performed by averaging the updated local weights received from the clients. Block 710 updates the prototypes as described above, with clustering being performed and a centroid being selected.

If block 712 determines that the number of global epochs T has not been reached yet, processing returns to block 704 and the updated global weights and client-specific prototypes are distributed to the clients 102. If not, the final global weights and client-specific prototypes are distributed and processing ends.

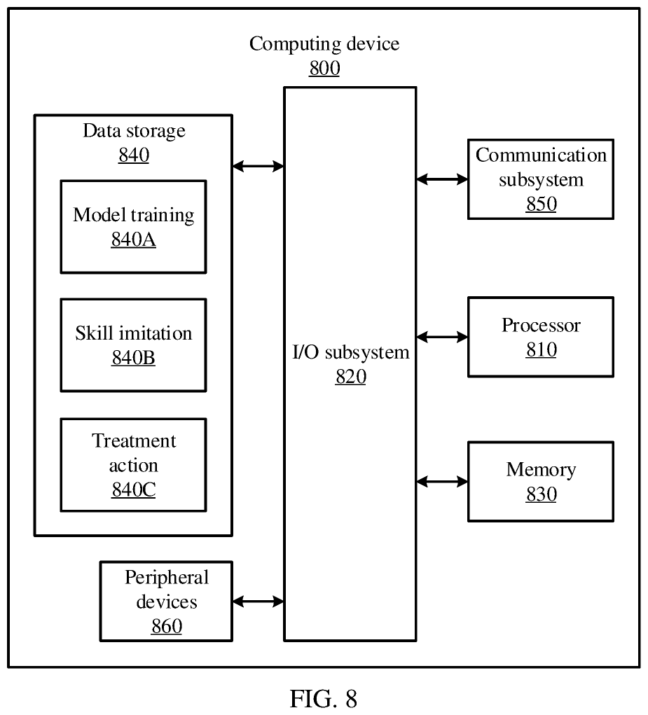

Referring now to FIG. 8, an exemplary computing device 800 is shown, in accordance with an embodiment of the present invention. The computing device 800 is configured to perform skill imitation.

The computing device 800 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 800 may be embodied as one or more compute sleds, memory sleds, or other racks, sleds, computing chassis, or other components of a physically disaggregated computing device.

As shown in FIG. 8, the computing device 800 illustratively includes the processor 810, an input/output subsystem 820, a memory 830, a data storage device 840, and a communication subsystem 850, and/or other components and devices commonly found in a server or similar computing device. The computing device 800 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 830, or portions thereof, may be incorporated in the processor 810 in some embodiments.

The processor 810 may be embodied as any type of processor capable of performing the functions described herein. The processor 810 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 830 may be embodied as any type of volatile or non-volatile memory or data storage capable of performing the functions described herein. In operation, the memory 830 may store various data and software used during operation of the computing device 800, such as operating systems, applications, programs, libraries, and drivers. The memory 830 is communicatively coupled to the processor 810 via the I/O subsystem 820, which may be embodied as circuitry and/or components to facilitate input/output operations with the processor 810, the memory 830, and other components of the computing device 800. For example, the I/O subsystem 820 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 820 may form a portion of a system-on-a-chip (SOC) and be incorporated, along with the processor 810, the memory 830, and other components of the computing device 800, on a single integrated circuit chip.

The data storage device 840 may be embodied as any type of device or devices configured for short-term or long-term storage of data such as, for example, memory devices and circuits, memory cards, hard disk drives, solid state drives, or other data storage devices. The data storage device 840 can store program code 840A for training a model, 840B for predicting an event, and/or 840C for performing a corrective action responsive to the predicted event. Any or all of these program code blocks may be included in a given computing system. The communication subsystem 850 of the computing device 800 may be embodied as any network interface controller or other communication circuit, device, or collection thereof, capable of enabling communications between the computing device 800 and other remote devices over a network. The communication subsystem 850 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 800 may also include one or more peripheral devices 860. The peripheral devices 860 may include any number of additional input/output devices, interface devices, and/or other peripheral devices. For example, in some embodiments, the peripheral devices 860 may include a display, touch screen, graphics circuitry, keyboard, mouse, speaker system, microphone, network interface, and/or other input/output devices, interface devices, and/or peripheral devices.

Of course, the computing device 800 may also include other elements (not shown), as readily contemplated by one of skill in the art, as well as omit certain elements. For example, various other sensors, input devices, and/or output devices can be included in computing device 800, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 800 are readily contemplated by one of ordinary skill in the art given the teachings of the present invention provided herein.

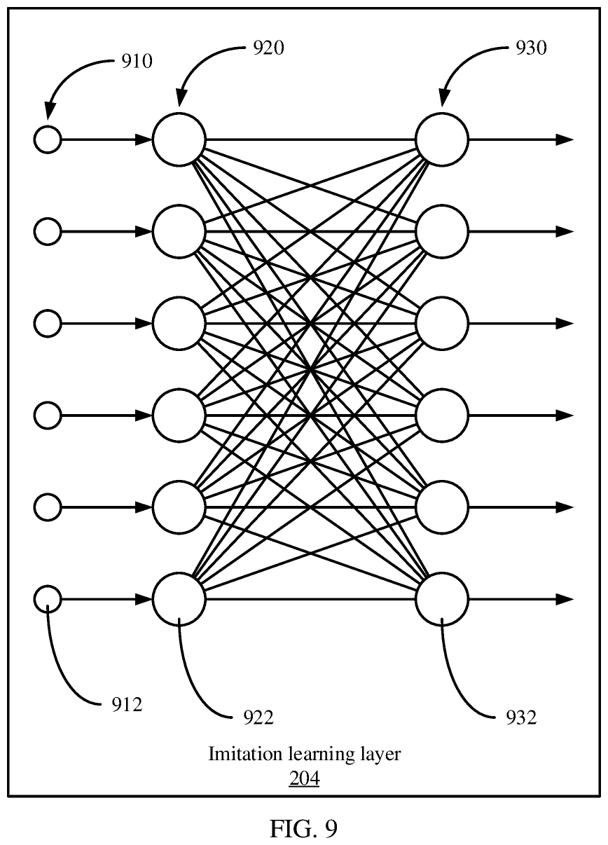

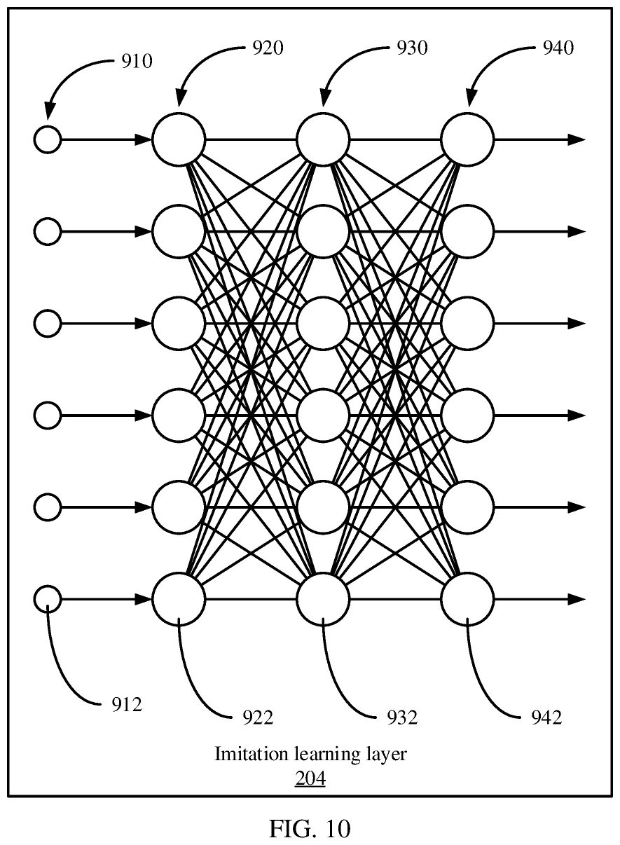

Referring now to FIGS. 9 and 10, exemplary neural network architectures are shown, which may be used to implement parts of the present models, such as the imitation learning layer 204. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 920 of source nodes 922, and a single computation layer 930 having one or more computation nodes 932 that also act as output nodes, where there is a single computation node 932 for each possible category into which the input example could be classified. An input layer 920 can have a number of source nodes 922 equal to the number of data values 912 in the input data 910. The data values 912 in the input data 910 can be represented as a column vector. Each computation node 932 in the computation layer 930 generates a linear combination of weighted values from the input data 910 fed into input nodes 920, and applies a non-linear activation function that is differentiable to the sum. The exemplary simple neural network can perform classification on linearly separable examples (e.g., patterns).

A deep neural network, such as a multilayer perceptron, can have an input layer 920 of source nodes 922, one or more computation layer(s) 930 having one or more computation nodes 932, and an output layer 940, where there is a single output node 942 for each possible category into which the input example could be classified. An input layer 920 can have a number of source nodes 922 equal to the number of data values 912 in the input data 910. The computation nodes 932 in the computation layer(s) 930 can also be referred to as hidden layers, because they are between the source nodes 922 and output node(s) 942 and are not directly observed. Each node 932, 942 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 932 in the one or more computation (hidden) layer(s) 930 perform a nonlinear transformation on the input data 912 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for training a healthcare treatment machine learning model, comprising:
aggregating local weights from a plurality of clients to update a set of global weights for an imitation-based skill learning model;
clustering a set of local prototype vectors from the plurality of clients to generate a plurality of clusters;
selecting representative vectors for the plurality of clusters as a set of global prototypes;
determining client-specific prototype vectors for the plurality of clients based on the representative vectors; and
distributing the updated set of global weights and the client-specific prototype vectors to the plurality of clients.

2. The method of claim 1, wherein the set of global weights includes weights of a convolution layer and weights of an imitation learning layer.

3. The method of claim 2, wherein the imitation learning layer implements an action-selection policy based on behavior cloning.

4. The method of claim 1, wherein selecting the representative vectors includes determining respective centroids of the plurality of clusters.

5. The method of claim 1, further comprising learning the local weights and the local prototype vectors at the plurality of clients based on initial global weights and initial prototypes.

6. The method of claim 5, wherein the learning includes minimizing an objective function that includes an imitation loss and a plurality of regularization losses.

7. The method of claim 6, wherein the plurality of regularization losses include a loss that regularizes a segment representation from the imitation-based skill learning model to be as adjacent to a closest prototype as possible, a loss that reverse-regularizes prototype vectors to be as similar to a segment representation as possible, and a loss that enforces a diverse structure of learnable parameterized prototype vectors to avoid redundancy and to improve generalizability of resulting prototypes.

8. The method of claim 1, wherein the local prototype vectors correspond to treatment actions that can be performed in a medical context.

9. The method of claim 8, further comprising:
measuring a patient's state information;
selecting a treatment action based on a skill predicted by the imitation-based skill learning model, based on the measured state information; and
notifying a medical professional of the treatment action to assist the medical professional in decision-making for patient management.

10. The method of claim 9, wherein the treatment action includes an instruction to a treatment system to automatically administer a treatment to a patient.

11. A system for training a healthcare treatment machine learning model, comprising:
a hardware processor; and
a memory that stores a computer program which, when executed by the hardware processor, causes the hardware processor to:
aggregate local weights from a plurality of clients to update a set of global weights for an imitation-based skill learning model;
cluster a set of local prototype vectors from the plurality of clients to generate a plurality of clusters;
select representative vectors for the plurality of clusters as a set of global prototypes;
determine client-specific prototype vectors for the plurality of clients based on the representative vectors; and
distribute the updated set of global weights and the client-specific prototype vectors to the plurality of clients.

12. The system of claim 11, wherein the set of global weights includes weights of a convolution layer and weights of an imitation learning layer.

13. The system of claim 12, wherein the imitation learning layer implements an action-selection policy based on behavior cloning.

14. The system of claim 11, wherein the computer program further causes the hardware processor to determine respective centroids of the plurality of clusters.

15. The system of claim 11, wherein the computer program further causes the hardware processor to trigger learning of the local weights and the local prototype vectors at the plurality of clients based on initial global weights and initial prototypes.

16. The system of claim 15, wherein the learning includes minimization of an objective function that includes an imitation loss and a plurality of regularization losses.

17. The system of claim 16, wherein the plurality of regularization losses include a loss that regularizes a segment representation from the imitation-based skill learning model to be as adjacent to a closest prototype as possible, a loss that reverse-regularizes prototype vectors to be as similar to a segment representation as possible, and a loss that enforces a diverse structure of learnable parameterized prototype vectors to avoid redundancy and to improve generalizability of resulting prototypes.

18. The system of claim 11, wherein the local prototype vectors correspond to treatment actions that can be performed in a medical context.

19. The system of claim 18, wherein the computer program further causes the hardware processor to:
measure a patient's state information;
select a treatment action based on a skill predicted by the imitation-based skill learning model, based on the measured state information; and
notify a medical professional of the treatment action to assist the medical professional in decision-making for patient management.

20. The system of claim 19, wherein the treatment action includes an instruction to a treatment system to automatically administer a treatment to a patient.
